(19) United States
(12) Patent Application Publication
(10) Pub. No.: US 2021/0147885 A1
(43) Pub. Date: May 20, 2021

(54) SELECTED ACETYL-COA SYNTHASE ENZYMES FOR REDUCTION OF ACETATE PRODUCTION IN YEAST

(71) Applicant: DANISCO US INC, PAlo Alto, CA (US)

(72) Inventor: Andrei Miasnikov, Union City, CA (US)

(21) Appl. No.: 16/606,105

(22) PCT Filed: Apr. 11, 2018
(86) PCT No.: PCT/US2018/027100

§ 371 Date: Oct. 17, 2019

Related U.S. Application Data
(60) Provisional application No. 62/486,178, filed on Apr. 17, 2017.

(51) Int. Cl. C12P 7/54; C12N 9/00

(52) U.S. Cl. CPC C12P 7/54; C12Y 602/01001; C12N 9/93

(57) ABSTRACT

Described is yeast having reduced acetate production as a consequence of expressing selected heterologous acetyl-Co synthase (ACS) enzymes. Such yeast may further be modified to reduce glycerol and/or increased ethanol production. The yeast is useful for producing ethanol from carbohydrate-containing substrates.

### TECHNICAL FIELD

The present compositions and methods relate to yeast having reduced acetate production as a consequence of expressing selected acetyl-Co synthase (ACS) enzymes. Such yeast may also be modified for reduced glycerol and/or increased ethanol production. The yeast is useful for producing ethanol from carbohydrate-containing substrates.

### BACKGROUND

Yeast-based ethanol production is based on the conversion of sugars into ethanol. The current annual fuel ethanol production by this method is about 90 billion liters worldwide. It is estimated that about 70% of the cost of ethanol production is the feedstock. Since the ethanol production volume is so large, even small yield improvements have massive economic impact for the industry. The conversion of one mole of glucose into two moles of ethanol and two moles of carbon dioxide is redox-neutral, with the maximum theoretical yield being about 51%. The current industrial yield is about 45%; therefore, there are opportunities to increase ethanol production.

Carbon dioxide, glycerol and yeast biomass are the major by-products of ethanol fermentation. During yeast growth and fermentation, a surplus of NADH is generated, which is used to produce glycerol for the purposes of redox balance and osmotic protection. Glycerol is considered a low value product and several approaches have been taken to reduce glycerol production.

Engineered yeast cells having a heterologous phosphoketolase pathway have been previously described (e.g., WO2015148272). These cells express heterologous phosphoketolase (PKL; EC 4.1.2.9) and phosphotransacetylase (PTA; EC 2.3.1.8), optionally with other enzymes, to channel carbon flux away from the glycerol pathway and toward the synthesis of acetyl-coA, which is then converted to ethanol. These cells are capable of increased ethanol production in a fermentation process when compared to otherwise-identical parent yeast cells. Unfortunately, such modified also produce increased acetate.

Acetate is toxic to most microorganisms including yeast. Wild-type yeast produces only minor amounts of acetate (e.g., 100-300 mg/l in a medium containing 60 g/l glucose). Acetate production in yeast expressing PKL is greatly increased (e.g., 1-2g/l in the same medium). Under conditions of industrial grain ethanol production where total amount of fermented glucose reaches 300 g/l, acetate production by PKL-expressing yeast strains can be extrapolated to 5-10 g/l. In reality, such concentrations are not observed because the cells stop growing due to acetate poisoning.

Several researchers have tried to over-express acetyl-Co synthase (ACS) to reduce acetate accumulation in PKL-expressing yeast. For, example, baker's yeast contains two ACS isoenzymes ACS1 and ACS2. ACS1 is under tight post-translational control being subject to catabolite inactivation in the presence of glucose (De Jong-Gubbels, P. et al. (1997) FEMS Microbiology Letters 153:75-81) while ACS2 has about 30-fold higher Km for acetate than ACS1 (van den Berg et al. (1996) J. Biol.Chem. 271:28953-59). In a study specifically aiming at reducing acetate production in yeast, de Jong-Gubbels et al. concluded that over-expression of either of the two native Saccharomyces cerevisiae ACS fails to decrease acetate level in yeast culture media ((1998) FEMS Microbiology Letters 165:15-20). In later, applied studies, attempts were made to use either native ACSI enzyme or a mutant (L614P) variant of Salmonella typimurium ACS that was reported to be resistant to catabolite inactivation. While the S. typhimurium enzyme preformed somewhat better than ACSI, acetate reduction was only modest.

In addition to being produced by certain yeast, some feedstocks used for the production of ethanol naturally contain acetate, which has the same detrimental effect on yeast. Accordingly, for various reasons, the need exists to modify yeast metabolic pathways to maximize ethanol production, while not increasing the production of undesirable pathway by-products such as acetate.

### SUMMARY

The present compositions and methods relate to yeast exhibiting reduced acetate production due to the expression of selected acetyl-Co synthase (ACS) enzymes. Aspects and embodiments of the compositions and methods are described in the following, independently-numbered paragraphs.

1. In one aspect, modified yeast cells are provided, comprising:

(i) an exogenous gene encoding an acetyl-Co synthase enzyme derived from an archaeal microorganism belonging to the genus Methanosaeta,

(ii) an exogenous gene encoding a low-affinity acetyl-Co synthase enzyme derived from ascomycete yeast,

(iii) an overexpressed endogenous low-affinity acetyl-CoA synthase, and/or

(iv) an exogenous gene encoding a functionally similar protein having at least 80% amino acid sequence identity to a polypeptide of SEQ ID NO: 4, SEQ ID NO:6 or SEQ ID NO: 10, or an active fragment thereof,

wherein the modified cells produce a reduced amount of acetate and/or grow in the presence of an increased amount of acetate, compared to otherwise identical yeast cells not comprising the exogenous gene.

2. In some embodiments, the modified yeast cells of paragraph 1 further comprise a genetic modification that, in the absence of the exogenous gene, causes the yeast cells to produce an increased amount of acetate.

3. In some embodiments of the modified yeast cells of paragraph 2, the genetic modification is the introduction of one or more genes of the phosphoketolase pathway or the deletion or disruption of genes of the glycerol synthesis pathway.

4. In some embodiments of the modified yeast cells of any of the preceding paragraphs, the reduction in the amount of acetate produced is at least 20%, at least 30% or at least 40%.

5. In some embodiments of the modified yeast cells of any of the preceding paragraphs, the functionally similar protein has at least 90%, at least 95% or at least 97% amino acid sequence identity to a polypeptide of SEQ ID NO: 3 or SEQ ID NO: 9, or an active fragment thereof.

6. In some embodiments, the modified yeast cells of any of the preceding paragraphs further comprise a gene encoding a carbohydrate processing enzyme or other protein of interest.

7. In another aspect, a method for reducing the amount of acetate produced by yeast cells during a fermentation of a starch hydrolysate is provided, comprising, introducing into the yeast:

(i) an exogenous gene encoding an acetyl-Co synthase enzyme derived from an archaeal microorganism belonging to the genus Methanosaeta,

(ii) an exogenous gene encoding a “low-affinity” acetyl-Co synthase enzyme derived from ascomycete yeast,

(iii) a genetic alteration that causes the overexpression of an endogenous low-affinity acetyl-CoA synthase, and/or

(iv) an exogenous gene encoding a functionally similar protein having at least 80% amino acid sequence identity to a polypeptide of SEQ ID NO: 3 or SEQ ID NO: 9, or an active fragment thereof.

8. In some embodiments of the method of paragraph 7, the yeast cells further comprise a genetic modification that, in the absence of the exogenous gene, would cause the yeast cells to produce an increased amount of acetate.

9. In some embodiments of the method of paragraph 8, the genetic modification is the introduction of genes of the phosphoketolase pathway or the deletion or disruption of genes of the glycerol synthesis pathway.

10. In some embodiments of the method of any of paragraphs 7-9, the reduction in the amount of acetate produced is at least 20%, at least 30% or at least 40%.

11. In some embodiments of the method of any of paragraphs 7-10, the functionally similar protein has at least 90%, at least 95% or at least 97% amino acid sequence identity to a polypeptide of SEQ ID NO: 3 or SEQ ID NO: 9, or an active fragment thereof.

12. In another aspect, modified yeast cells produced by the method of any of paragraphs 7-11 is provided.

These and other aspects and embodiments of present modified cells and methods will be apparent from the description, including the accompanying Figures.

### BRIEF DESCRIPTION OF THE DRAWINGS

FIG. 1 is a functional map of vector pX(DeltaTDH ACSSalty), the prototype for all constructs used to express various acetyl-CoA synthases described, herein.

FIG. 2 is a functional map of vector pPATH1(TDH_A2_ADE2). This vector carries three expression cassettes for phosphoketolase from Bifidobacterium animalis, phosphotransacetylase from Lactobacillus plantarum and acylating acetaldehyde dehydrogenase from Salmonella enterica. Together these three enzymes form a metabolic pathway referred to as the “phosphoketolase pathway.”

FIG. 3 is a graph showing reduced acetate production in derivatives of high-acetate-producing-strain A2 expressing various homologous and heterologous ACS enzymes. Strain FERMAXGOLD® is the parental strain of A2 and produces acetate in amounts typical for wild-type yeast strains.

### DETAILED DESCRIPTION

### I. Definitions

Prior to describing the present compositions and methods in detail, the following terms are defined for clarity. Terms not defined should be accorded their ordinary meanings as used in the relevant art.

As used herein, the term “gene” is synonymous with the term “allele” in referring to a nucleic acid that encodes and directs the expression of a protein or RNA.

As used herein, the terms “wild-type” and “native” are used interchangeably and refer to genes, proteins, strains, and biochemical pathways found in nature.

As used herein, “deletion of a gene,” refers to its removal from the genome of a host cell. Deletion may be complete, meaning an entire gene (i.e., at least the entire coding sequences are removed) or partial, meaning that only a portion of the coding sequences or regulatory sequences are removed but which prevent the production of a functional gene product.

As used herein, “attenuation of a pathway” or “attenuation of the flux through a pathway” i.e., a biochemical pathway, refers broadly to any genetic or chemical manipulation that reduces or completely stops the flux of biochemical substrates or intermediates through a metabolic pathway. Attenuation of a pathway may be achieved by a variety of well-known methods. Such methods include but are not limited to: complete or partial deletion of one or more genes, replacing wild-type alleles of these genes with mutant forms encoding enzymes with reduced catalytic activity or increased Km values, modifying the promoters or other regulatory elements that control the expression of one or more genes, engineering the enzymes or the mRNA encoding these enzymes for a decreased stability, misdirecting enzymes to cellular compartments where they are less likely to interact with substrate and intermediates, the use of interfering RNA, and the like.

As used herein, “disruption of a gene” refers broadly to any genetic manipulation that substantially prevents a cell from producing a functional gene product. Exemplary methods of gene disruption include complete or partial deletion of a gene and making mutations in coding or regulatory sequences.

As used herein, the terms “genetic manipulation” and “genetic alteration” are used interchangeably and refer to the alteration/change of a nucleic acid sequence. The alteration can be included but is not limited to a substitution, deletion, insertion or chemical modification of at least one nucleic acid in the nucleic acid sequence.

As used herein, “expressing a polypeptide” and similar terms, refer to the cellular process of producing a polypeptide using the translation machinery (e.g., ribosomes) of the cell.

As used herein, “overexpressing a polypeptide,” “increasing the expression of a polypeptide,” and similar terms, refer to expressing a polypeptide at higher-than-normal levels compared to those observed with parental or “wild-type cells that do not include a specified genetic modification. Overexpression can be accomplished by using a stronger promoter with an endogenous gene and/or introducing additional copies of a gene into a cell, e.g., in the form of an additional expression cassette, using a suitable promoter.

As used herein, an “expression cassette” refers to a nucleic acid that includes an amino acid coding sequence, promoters, terminators, and other nucleic acid sequence needed to allow the encoded polypeptide to be produced in a cell. Expression cassettes can be exogenous (i.e., introduced into a cell) or endogenous (i.e., extant in a cell).

As used herein, “anaerobic fermentation” refers to growth in the absence of oxygen.

As used herein, “aerobic fermentation” refers to growth in the presence of oxygen.

As used herein, the terms “polypeptide” and “protein” (and/or their respective plural forms) are used interchangeably to refer to polymers of any length comprising amino acid residues linked by peptide bonds. The conventional one-letter or three-letter codes for amino acid residues are used herein. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, functionally and/or structurally similar proteins are considered to be “related proteins.” Such proteins can be derived from organisms of different genera and/or species, or even different classes of organisms (e.g., bacteria and fungi). Related proteins also encompass homologs determined by primary sequence analysis, determined by secondary or tertiary structure analysis, or determined by immunological cross-reactivity.

As used herein, the term “percent amino acid sequence identity,” or similar, means that a particular sequence has at least a certain percentage of amino acid residues identical to those in a specified reference sequence, when aligned using the CLUSTAL W algorithm with default parameters. See Thompson et al. (1994) Nucleic Acids Res. 22:4673-80. Default parameters for the CLUSTAL W algorithm are:

| | |
|---|---|
| Gap opening penalty: | 10.0 |
| Gap extension penalty: | 0.05 |
| Protein weight matrix: | BLOSUM series |
| DNA weight matrix: | IUB |
| Delay divergent sequences %: | 40 |
| Gap separation distance: | 8 |
| DNA transitions weight: | 0.50 |
| List hydrophilic residues: | GPSNDQEKR |
| Use negative matrix: | OFF |
| Toggle Residue specific penalties: | ON |
| Toggle hydrophilic penalties: | ON |
| Toggle end gap separation penalty | OFF |

As used herein, the singular articles “a,” “an,” and “the” encompass the plural referents unless the context clearly dictates otherwise. All references cited herein are hereby incorporated by reference in their entirety. The following abbreviations/acronyms have the following meanings unless otherwise specified:

EC enzyme commission

kDa kiloDalton

kb kilobase

MW molecular weight

w/v weight/volume

w/w weight/weight

v/v volume/volume

wt % weight percent

° C. degrees Centigrade

H₂O water

H₂O₂ hydrogen peroxide

dH₂O or DI deionized water

dIH₂O deionized water, Milli-Q filtration

g or gm gram

82 g microgram

mg milligram

kg kilogram

lb pound

mL and μl microliter

mL and ml milliliter

mm millimeter

μm micrometer

mol mole

mmol millimole

M molar

mM millimolar

μM micromolar

nm nanometer

U unit

ppm parts per million

sec and ″ second

min and ′ minute

hr and h hour

EtOH ethanol

eq. equivalent

PCR polymerase chain reaction

DNA deoxyribonucleic acid

Δ relating to a deletion

bp base pairs

### II. Yeast Expressing Selected Acetyl-Co Synthase Enzymes

The present compositions and methods relate to the use of selected acetyl-Co synthase (ACS) enzymes to reduce the production of acetate in engineered yeast cells. The selected ACS were identified after screening a number of different taxonomically-divergent ACS. Several ACS outperformed any reported in the literature. An archaeal ACS from Methanosaeta conciliiACS was the overall winner while a low-affinity ACS from yeast Saccharomyces cerevisiae and Zygosaccharomyces rouxii (ACS2) also performed better than the most efficient ACS reported previously. The decrease in acetate production in yeast resulting from the introduction of an exogenous gene encoding one of these enzymes (or overexpressing an endogenous gene) is at least 10%, at least 20%, at least 30% and even at least 40%, compared to the amount of acetate produced by yeast that over-produces acetate.

Use of the selected acetyl-Co synthase (ACS) enzymes will reduce the production of acetate in yeast that produce an unwanted excess of acetate, including but not limited to engineered yeast such as those described by Miasnikov (WO2015148272) and Pronk et al. (WO2011010923), and/or reduce the amount of exogenous acetate naturally present in the fermentation medium.

III. Combination with additional mutations that affect alcohol production

In some embodiments the present modified cells may additionally express heterologous phosphoketolase (PKL; EC 4.1.2.9) and phosphotransacetylase (PTA; EC 2.3.1.8), optionally with other enzymes, to channel carbon flux away from the glycerol pathway and toward the synthesis of acetyl-coA, which is then converted to ethanol. An exemplary phosphoketolase can be obtained from Gardnerella vaginalis (UniProt/TrEMBL Accession No.: WP_016786789). An exemplary phosphotransacetylase can be obtained from Lactobacillus plantarum (UniProt/TrEMBL Accession No.: WP_003641060). In some embodiments the present modified cells may further have an artificial alternative pathway for making Ac-CoA that does not contribute to a redox cofactor imbalance in the cells under anaerobic conditions, as described by Miasnikov (WO2015148272).

The present modified cells may further include mutations that result in attenuation of the native glycerol biosynthesis pathway, which are known to increase alcohol production. Methods for attenuation of the glycerol biosynthesis pathway in yeast are known and include reduction or elimination of endogenous NAD-dependent glycerol 3-phosphate dehydrogenase (GPD) or glycerol phosphate phosphatase activity (GPP), for example by disruption of one or more of the genes GPD1, GPD2, GPP1 and/or GPP2. See, e.g., U.S. Pat. Nos. 9,175,270 (Elke et al.), 8,795,998 (Pronk et al.) and 8,956,851 (Argyros et al.).

In some embodiments the present modified cells may further include a heterologous gene encoding a protein with NAD+-dependent acetylating acetaldehyde dehydrogenase activity and/or a heterologous gene encoding a pyruvate-formate lyase. The introduction of such genes in combination with attenuation of the glycerol pathway is described, e.g., in U.S. Pat. No. 8,795,998 (Pronk et al.). However, in most embodiments of the present compositions and methods, the introduction of an acetylating acetaldehyde dehydrogenase and/or a pyruvate-formate lyase is not required because the need for these activities is obviated by the attenuation of the native biosynthetic pathway for making acetyl-CoA that contributes to redox cofactor imbalance.

In some embodiments the present modified cells may further have an attenuated native biosynthetic pathway in yeast for making Ac-CoA, which contributes to redox cofactor imbalance in the cells under anaerobic conditions and thus requires glycerol production for restoring the redox balance. In some embodiments, the compositions and methods involve disruption of one, several or all the native genes (e.g., ALD2 ALD3 ALD4 ALDS and ALD6) encoding aldehyde dehydrogenase (EC 1.2.1.3). The native yeast Ac-CoA pathway, including aldehyde dehydrogenase, is well described in the literature. Deletion of these native genes has been described in, e.g., Kozak et al. (2014) Metabolic Engineering 21:46-59).

In some embodiments, the present modified yeast cells further comprise a butanol biosynthetic pathway. In some embodiments, the butanol biosynthetic pathway is an isobutanol biosynthetic pathway. In some embodiments, the isobutanol biosynthetic pathway comprises a polynucleotide encoding a polypeptide that catalyzes a substrate to product conversion selected from the group consisting of: (a) pyruvate to acetolactate; (b) acetolactate to 2,3-dihydroxyisovalerate; (c) 2,3 -dihydroxyisovalerate to 2-ketoisovalerate; (d) 2-ketoisovalerate to isobutyraldehyde; and (e) isobutyraldehyde to isobutanol. In some embodiments, the isobutanol biosynthetic pathway comprises polynucleotides encoding polypeptides having acetolactate synthase, keto acid reductoisomerase, dihydroxy acid dehydratase, ketoisovalerate decarboxylase, and alcohol dehydrogenase activity.

In some embodiments, the modified yeast cells comprising a butanol biosynthetic pathway further comprise a modification in a polynucleotide encoding a polypeptide having pyruvate decarboxylase activity. In some embodiments, the yeast cells comprise a deletion, mutation, and/or substitution in an endogenous polynucleotide encoding a polypeptide having pyruvate decarboxylase activity. In some embodiments, the polypeptide having pyruvate decarboxylase activity is selected from the group consisting of: PDC1, PDC5, PDC6, and combinations thereof. In some embodiments, the yeast cells further comprise a deletion, mutation, and/or substitution in one or more endogenous polynucleotides encoding FRA2, ALD6, ADH1, GPD2, BDH1, and YMR226C.

In some embodiments, the present modified cells include any number of additional genes of interest encoding proteins of interest in addition to the ACS enzyme. Proteins of interest, include selectable markers, carbohydrate-processing enzymes, and other commercially-relevant polypeptides, including but not limited to an enzyme selected from the group consisting of a dehydrogenase, a transketolase, a phosphoketolase, a transladolase, an epimerase, a phytase, a xylanase, a β-glucanase, a phosphatase, a protease, an α-amylase, a β-amylase, a glucoamylase, a pullulanase, an isoamylase, a cellulase, a trehalase, a lipase, a pectinase, a polyesterase, a cutinase, an oxidase, a transferase, a reductase, a hemicellulase, a mannanase, an esterase, an isomerase, a pectinases, a lactase, a peroxidase and a laccase. Proteins of interest may be secreted, glycosylated, and otherwise modified.

### IV. Use of the Modified Yeast for Increased Alcohol Production

The present compositions and methods include methods for increasing alcohol production using the modified yeast in fermentation reactions. Such methods are not limited to a particular fermentation process. The present engineered yeast is expected to be a “drop-in” replacement for convention yeast in any alcohol fermentation facility. While primarily intended for fuel ethanol production, the present yeast can also be used for the production of potable alcohol, including wine and beer.

### V. Yeast Cells Suitable for Modification

Yeasts are unicellular eukaryotic microorganisms classified as members of the fungus kingdom and include organisms from the phyla Ascomycota and Basidiomycota. Yeast that can be used for alcohol production include, but are not limited to, Saccharomyces spp., including S. cerevisiae, as well as Kluyveromyces, Lachancea and Schizosaccharomyces spp. Numerous yeast strains are commercially available, many of which have been selected or genetically engineered for desired characteristics, such as high alcohol production, rapid growth rate, and the like. Some yeasts have been genetically engineered to produce heterologous enzymes, such as glucoamylase or α-amylase.

### VI. Carbohydrate Substrates and Processes

Ethanol production from a number of carbohydrate substrates is well known, as are innumerable variations and improvements to enzymatic and chemical conditions and mechanical processes. The present compositions and methods are believed to be fully compatible with such substrates and conditions.

### EXAMPLES

### Example 1. Construction of Expression Vectors for Testing Acetyl-CoA Synthases

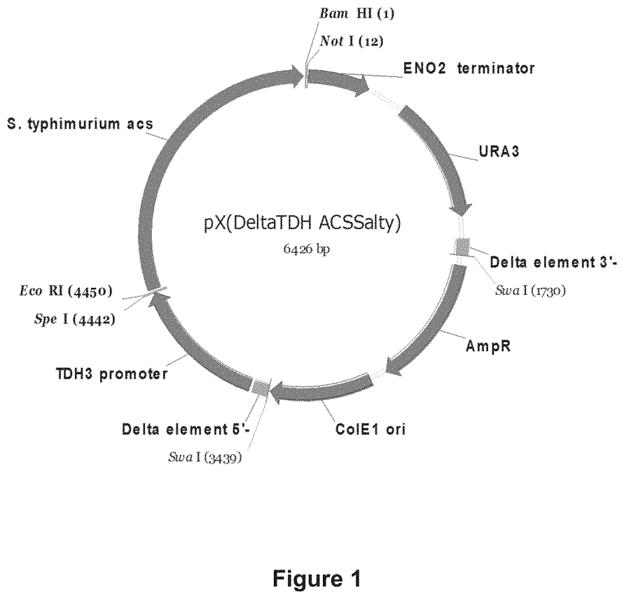

The coding regions of the S. cerevisiae genes ACS1 and ACS2 as well as Z. rouxii gene ACS2 were amplified by PCR using chromosomal DNA of the corresponding hosts as templates. The genes encoding the Salmonella typhimurium acs mutant L641P and the gene encoding the Methanosaeta concilii acsA1 were synthesized based on known amino acid sequence. The codon composition of the synthetic genes was optimized to correspond to the codon bias of the highly-expressed genes of S. cerevisiae. Nucleotide and amino acid sequences of all the genes encoding various ACS of this study are listed as SEQ ID NO: 1-10. Vector pX(DeltaTDH_ACSSalty) (FIG. 1) was assembled using conventional genetic engineering methods. All yeast-derived elements in this vector, i.e., the THD3 promoter and ENO2 transcription terminator, 3′ and 5′ sequences of the yeast β-element, and the URA3 selectable marker, were derived from yeast chromosomal DNA. Bacterial sequences (i.e., the colE1 replication origin and ampicillin resistance gene) were derived from the well-known pUC19 vector. The vectors for expression of other ACSs were identical to pX(DeltaTDH_ACSSalty) except that the coding sequence of S. typhimurium ACS L641P variant was replaced with the sequences of SEQ ID NO: 1, 3, 5 or 9.

### Example 2. Testing the Effect of Different ACS on Acetate Production in Yeast

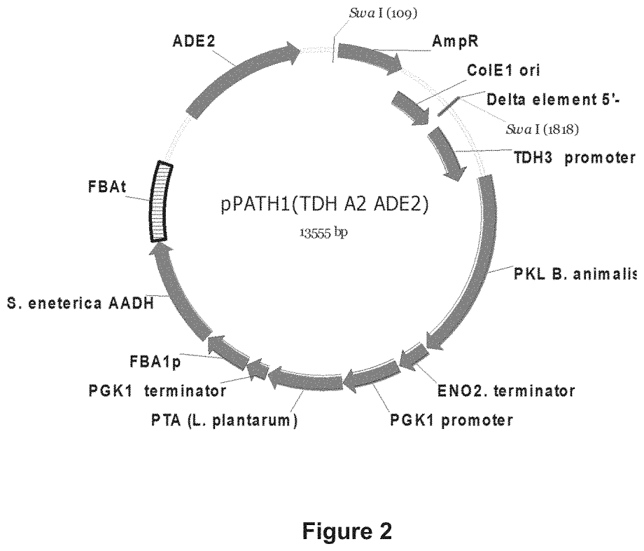

For testing the ability of different ACS to help yeast assimilate excessive acetate, a test strain of yeast that over-produces acetate was used. This strain, herein referred to as A2, was obtained by transforming a ura3, ade2 mutant of the commercially-available S. cerevisiae strain FERMAXGOLD® with the large SwaI fragment of vector PATH1(TDH_A2_ADE2) (FIG. 2). Strain A2 expresses three enzymes: phosphoketolase, phosphotransacetylase and acylating acetaldehyde dehydrogenase making up the “phosphoketolase pathway.” Strain A2 produces significantly higher level of acetate than the wild-type precursor strain FERMAXGOLD®. The reasons for elevated acetate production in strain A2 are, firstly, that acetyl-phosphate, a product of phosphoketolase reaction, is chemically unstable and can undergo spontaneous hydrolysis into acetic and phosphoric acids and, secondly, acetyl-CoA formed in the phosphoketolase pathway reduces the metabolic demand for synthesizing it from acetic acid (the metabolic pathway yeast normally uses for production of cytosolic Ac-CoA anaerobically).

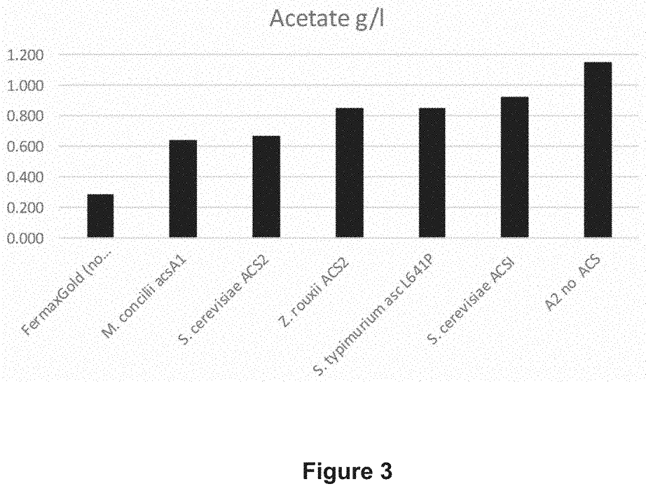

Strain A2, which is an uracil auxotroph, was transformed to uracil prototrophy with a series of five vectors directing the expression of five different ACS. Except for the ACS coding sequences, the vectors were identical. The prototype vector is pX(DeltaTDH_ACSSalty) expressing S. typhimurium acs L641P variant (FIG. 1). In all cases the large SwaI fragment of the respective vector was used as the transforming DNA (including all the functional yeast elements but excluding the sequences needed for propagation of the vector in bacteria). Eight, randomly-selected transformants from each of the five transformations were selected and cultivated in a medium containing 60 g/l glucose, 0.67 g/l yeast nitrogen base without amino acids and ammonium sulfate, 2 g/l urea. The cultivations were performed for 48 hours at 32° C. with shaking. The culture media was then analyzed for fermentation products by HPLC. The results of these experiments are summarized in Table 1 and the graph in FIG. 3.

Surprisingly, the over-expression of ACS2 from S. cerevisiae, encoding the “low affinity” ACS, efficiently reduces acetate accumulation in culture medium. Such observations contradict published literature describing that over-expression of either ACS1 nor ACS2 does not have an appreciable effect on acetate accumulation in yeast cultures (de Jong-Gubbels et al. (1998) FEMS Microbiology Letters 165:15-20). ACS2 from osmophilic yeast, Z. rouxii, was less efficient than its counterpart from S. cerevisiae but still performed slightly better than “industry standard” ACS previously described as useful for reduced acetate production in yeast (i.e., a L641P mutant form of ACS from S. typhimurium). The overall winner of the study was the enzyme encoded by acsA1 gene of M concilii, an archaeal microorganism from soil known to utilize acetate as the preferred carbon source.

TABLE 1
Acetate production by high acetate strain
A2 and its ACS-expressing derivatives

| | Acetate (g/L) |
|---|---|
| Strain | Average of 8 cultures |
| FERMAXGOLD ® (no PKL pathway) | 0.286 |
| A2 expressing Methanosaeta concilii acsA1 | 0.641 |
| A2 expressing S. cerevisiae ACS2 | 0.666 |
| A2 expressing Z. rouxii ACS2 | 0.849 |
| A2 expressing S. typimurium asc L641P | 0.852 |
| A2 expressing S. cerevisiae ACSI | 0.925 |
| A2 no ACS | 1.150 |

### SEQUENCES

Polyucleotide sequence of the ACSI gene from S. cerevisiae strain FERMAXGOLD® (SEQ ID NO: 1):

ATGTCGCCCTCTGCCGTACAATCATCAAAACTAGAAGAACAGTCAAGTGA
AATTGACAAGTTGAAAGCAAAAATGTCCCAGTCTGCCTCCACTGCGCAGC
AGAAGAAGGAACATGAGTATGAACATTTGACCTCGGTCAAGATCGTGCCA
CAACGGCCCATCTCAGATAGACTGCAGCCCGCAATTGCTACCCACTATTC
TCCACACTTGGACGGGTTGCAGGACTATCAGCGCTTGCACAAGGAGTCTA
TTGAAGACCCTGCTAAGTTCTTCGGTTCTAAAGCTACCCAATTTTTAAAC
TGGTCTAAGCCATTCGATAAGGTGTTCATCCCAGACCCTAAAACGGGCAG
GCCCTCCTTCCAGAACAATGCATGGTTCCTCAACGGCCAATTAAACGCCT
GTTACAACTGTGTTGACAGACATGCCTTGAAGACCCCTAACAAGAAAGCC
ATTATTTTCGAAGGTGACGAGCCTGGCCAAGGCTATTCCATTACCTACAA
GGAACTACTTGAAGAAGTTTGTCAAGTGGCACAAGTGCTGACTTACTCTA
TGGGCGTTCGCAAGGGCGATACTGTTGCCGTGTACATGCCTATGGTCCCA
GAAGCAATCATAACCTTGTTGGCCATTTCCCGTATCGGTGCCATTCACTC
CGTAGTCTTTGCCGGGTTTTCTTCCAACTCCTTGAGAGATCGTATCAACG
ATGGGGACTCTAAAGTTGTCATCACTACAGATGAATCCAACAGAGGTGGT
AAAGTCATTGAGACTAAAAGAATTGTTGATGACGCGCTAAGAGAGACCCC
AGGCGTGAGACACGTCTTGGTTTATAGAAAGACCAACAATCCATCTGTTG
CTTTCCATGCCCCCAGAGATTTGGATTGGGCAACAGAAAAGAAGAAATAC
AAGACCTACTATCCATGCACACCCGTTGATTCTGAGGATCCATTATTCTT
GTTGTATACGTCTGGTTCTACTGGTGCCCCCAAGGGTGTTCAACATTCTA
CCGCAGGTTACTTGCTGGGAGCTTTGTTGACCATGCGCTACACTTTTGAC
ACTCACCAAGAAGACGTTTTCTTCACAGCTGGAGACATTGGCTGGATTAC
AGGCCACACTTATGTGGTTTATGGTCCCTTACTATATGGTTGTGCCACTT
TGGTCTTTGAAGGGACGCCTGCATACCCAAATTACTCCCGTTATTGGGAT
ATTATTGATGAACACAAAGTCACCCAATTTTATGTTGCGCCAACTGCTTT
GCGTTTGTTGAAAAGAGCTGGTGATTCCTACATCGAAAATCATTCCTTAA
AATCTTTGCGTTGCTTGGGTTCGGTCGGTGAGCCAATTGCTGCTGAAGTT
TGGGAGTGGTACTCTGAAAAAATAGGTAAAAATGAAATCCCCATTGTAGA
CACCTACTGGCAAACAGAATCTGGTTCGCATCTGGTCACCCCGCTGGCTG
GTGGTGTCACACCAATGAAACCGGGTTCTGCCTCATTCCCCTTCTTCGGT
ATTGATGCAGTTGTTCTTGACCCTAACACTGGTGAAGAACTTAACACCAG
CCACGCAGAGGGTGTCCTTGCCGTCAAAGCTGCATGGCCATCATTTGCAA
GAACTATTTGGAAAAATCATGATAGGTATCTAGACACTTATTTGAACCCT
TACCCTGGCTACTATTTCACTGGTGATGGTGCTGCAAAGGATAAGGATGG
TTATATCTGGATTTTGGGTCGTGTAGACGATGTGGTGAACGTCTCTGGTC
ACCGTCTGTCTACCGCTGAAATTGAGGCTGCTATTATCGAAGATCCAATT
GTGGCCGAGTGTGCTGTTGTCGGATTCAACGATGACTTGACTGGTCAAGC
AGTTGCTGCATTTGTGGTGTTGAAAAACAAATCTAGTTGGTCCACCGCAA
CAGATGATGAATTACAAGATATCAAGAAGCATTTGGTCTTTACTGTTAGA
AAAGACATCGGGCCATTTGCCGCACCAAAATTGATCATTTTAGTGGATGA
CTTGCCCAAGACAAGATCTGGCAAAATTATGAGACGTATTTTAAGAAAAA
TCCTAGCAGGAGAAAGTGACCAACTAGGCGACGTTTCTACATTGTCAAAC
CCTGGCATTGTTAGACATCTAATTGATTCGGTCAAGTTGTAA

Amino acid sequence of the ACS1p from S. cerevisiae strain FERMAXGOLD® (SEQ ID NO: 2):

MSPSAVQSSKLEEQSSEIDKLKAKMSQSASTAQQKKEHEYEHLTSVKIVP
QRPISDRLQPAIATHYSPHLDGLQDYQRLHKESIEDPAKFFGSKATQFLN
WSKPFDKVFIPDPKTGRPSFQNNAWFLNGQLNACYNCVDRHALKTPNKKA
IIFEGDEPGQGYSITYKELLEEVCQVAQVLTYSMGVRKGDTVAVYMPMVP
EAIITLLAISRIGAIHSVVFAGFSSNSLRDRINDGDSKVVITTDESNRGG
KVIETKRIVDDALRETPGVRHVLVYRKTNNPSVAFHAPRDLDWATEKKKY
KTYYPCTPVDSEDPLFLLYTSGSTGAPKGVQHSTAGYLLGALLTMRYTFD
THQEDVFFTAGDIGWITGHTYVVYGPLLYGCATLVFEGTPAYPNYSRYWD
IIDEHKVTQFYVAPTALRLLKRAGDSYIENHSLKSLRCLGSVGEPIAAEV
WEWYSEKIGKNEIPIVDTYWQTESGSHLVTPLAGGVTPMKPGSASFPFFG
IDAVVLDPNTGEELNTSHAEGVLAVKAAWPSFARTIWKNHDRYLDTYLNP
YPGYYFTGDGAAKDKDGYIWILGRVDDVVNVSGHRLSTAEIEAAIIEDPI
VAECAVVGFNDDLTGQAVAAFVVLKNKSSWSTATDDELQDIKKHLVFTVR
KDIGPFAAPKLIILVDDLPKTRSGKIMRRILRKILAGESDQLGDVSTLSN
PGIVRHLIDSVKL

Polynucleotide sequence of the ACS2 gene from S. cerevisiae strain FERMAXGOLD® (SEQ ID NO: 3):

ATGACAATCAAGGAACATAAAGTAGTTCATGAAGCTCACAACGTAAAGGC
TCTTAAGGCTCCTCAACATTTTTACAACAGCCAACCCGGCAAGGGTTACG
TTACTGATATGCAACATTATCAAGAAATGTATCAACAATCTATCAATGAG
CCAGAAAAATTCTTTGATAAGATGGCTAAGGAATACTTGCATTGGGATGC
TCCATACACCAAAGTTCAATCTGGTTCATTGAACAATGGTGATGTTGCAT
GGTTTTTGAACGGTAAATTGAATGCATCATACAATTGTGTTGACAGACAT
GCCTTTGCTAATCCCGACAAGCCAGCTTTGATCTATGAAGCTGATGACGA
ATCCGACAACAAAATCATCACATTTGGTGAATTACTCAGAAAAGTTTCCC
AAATCGCTGGTGTCTTAAAAAGCTGGGGCGTTAAGAAAGGTGACACAGTG
GCTATCTATTTGCCAATGATTCCAGAAGCGGTCATTGCTATGTTGGCTGT
GGCTCGTATTGGTGCTATTCACTCTGTTGTCTTTGCTGGGTTCTCCGCTG
GTTCGTTGAAAGATCGTGTCGTTGACGCTAATTCTAAAGTGGTCATCACT
TGTGATGAAGGTAAAAGAGGTGGTAAGACCATCAACACTAAAAAAATTGT
TGACGAAGGTTTGAACGGAGTCGATTTGGTTTCCCGTATCTTGGTTTTCC
AAAGAACTGGTACTGAAGGTATTCCAATGAAGGCCGGTAGAGATTACTGG
TGGCATGAGGAGGCCGCTAAGCAGAGAACTTACCTACCTCCTGTTTCATG
TGACGCTGAAGATCCTCTATTTTTGTTATACACTTCCGGTTCCACTGGTT
CTCCAAAGGGTGTCGTTCACACTACAGGTGGTTATTTATTAGGTGCCGCT
TTAACAACTAGATACGTTTTTGATATTCACCCAGAAGATGTTCTCTTCAC
TGCCGGTGACGTCGGCTGGATCACGGGTCACACCTATGCTCTATATGGTC
CATTAACCTTGGGTACCGCCTCAATAATTTTCGAATCCACTCCTGCCTAC
CCAGATTATGGTAGATATTGGAGAATTATCCAACGTCACAAGGCTACCCA
TTTCTATGTGGCTCCAACTGCTTTAAGATTAATCAAACGTGTAGGTGAAG
CCGAAATTGCCAAATATGACACTTCCTCATTACGTGTCTTGGGTTCCGTC
GGTGAACCAATCTCTCCAGACTTATGGGAATGGTATCATGAAAAAGTGGG
TAACAAAAACTGTGTCATTTGTGACACTATGTGGCAAACAGAGTCTGGTT
CTCATTTAATTGCTCCTTTGGCAGGTGCTGTCCCAACAAAACCTGGTTCT
GCTACCGTGCCATTCTTTGGTATTAACGCTTGTATCATTGACCCTGTTAC
AGGTGTGGAATTAGAAGGTAATGATGTCGAAGGTGTCCTTGCCGTTAAAT
CACCATGGCCATCAATGGCTAGATCTGTTTGGAACCACCACGACCGTTAC
ATGGATACTTACTTGAAACCTTATCCTGGTCACTATTTCACAGGTGATGG
TGCTGGTAGAGATCATGATGGTTACTACTGGATCAGGGGTAGAGTTGACG
ACGTTGTAAATGTTTCCGGTCATAGATTATCCACATCAGAAATTGAAGCA
TCCATCTCAAATCACGAAAACGTCTCGGAAGCTGCTGTTGTCGGTATTCC
AGATGAATTGACCGGTCAAACCGTCGTTGCATATGTTTCCCTAAAAGATG
GTTATCTACAAAACAACGCTACTGAAGGTGATGCAGAACACATCACACCA
GATAATTTACGTAGAGAATTGATCTTACAAGTTAGGGGTGAGATTGGTCC
TTTCGCCTCACCAAAAACCATTATTCTAGTTAGAGATCTACCAAGAACAA
GGTCAGGAAAGATTATGAGAAGAGTTCTAAGAAAGGTTGCTTCTAACGAA
GCCGAACAGCTAGGTGACCTAACTACTTTGGCCAACCCAGAAGTTGTACC
TGCCATCATTTCTGCTGTAGAGAACCAATTTTTCTCTCAAAAAAAGAAAT
AA

Amino acid sequence of the ACS2p from S. cerevisiae strain FERMAXGOLD® (SEQ ID NO: 4):

MTIKEHKVVHEAHNVKALKAPQHFYNSQPGKGYVTDMQHYQEMYQQSINE
PEKFFDKMAKEYLHWDAPYTKVQSGSLNNGDVAWFLNGKLNASYNCVDRH
AFANPDKPALIYEADDESDNKIITFGELLRKVSQIAGVLKSWGVKKGDTV
AIYLPMIPEAVIAMLAVARIGAIHSVVFAGFSAGSLKDRVVDANSKVVIT
CDEGKRGGKTINTKKIVDEGLNGVDLVSRILVFQRTGTEGIPMKAGRDYW
WHEEAAKQRTYLPPVSCDAEDPLFLLYTSGSTGSPKGVVHTTGGYLLGAA
LTTRYVFDIHPEDVLFTAGDVGWITGHTYALYGPLTLGTASIIFESTPAY
PDYGRYWRIIQRHKATHFYVAPTALRLIKRVGEAEIAKYDTSSLRVLGSV
GEPISPDLWEWYHEKVGNKNCVICDTMWQTESGSHLIAPLAGAVPTKPGS
ATVPFFGINACIIDPVTGVELEGNDVEGVLAVKSPWPSMARSVWNHHDRY
MDTYLKPYPGHYFTGDGAGRDHDGYYWIRGRVDDVVNVSGHRLSTSEIEA
SISNHENVSEAAVVGIPDELTGQTVVAYVSLKDGYLQNNATEGDAEHITP
DNLRRELILQVRGEIGPFASPKTIILVRDLPRTRSGKIMRRVLRKVASNE
AEQLGDLTTLANPEVVPAIISAVENQFFSQKKK

Nucleotide sequence of the ACS2 gene from Z. rouxii strain CBS762 (SEQ ID NO: 5)

ATGACAACTAAGGAACATAAGACCGTTCATGAGGATAAACCATTTACAAA
GACGAAGTTTGGTTCATTGGAAAATGGTGATACTACTTGGTTTTTAAACG
GTGAGTTGAATGCTGCTTACAACTGTGTTGATAGACATGCTTTTGCCAAT
CCTGATAAACCAGCATTGATCTACGAAGCGGATGAAGAAGCAGACAACAG
GGTTGTTACATTCGGTGAGCTTTTGAGACAAGTTTCTCAAGTTGCTGGTG
TTCTTCACAGCTGGGGGGTTAGAAAAGGTGATACCGTCGCAGTTTATCTA
CCAATGATTCCTGAAGCTGTTGTTGCAATGTTGGCCGTTGCCAGATTAGG
TGCAATTCATTCTGTTGTTTTTGCAGGTTTTTCAGCAGGTTCCTTGAAGG
ATCGTGTTGTAGACGCAGGTTGTAAAGTTGTTATTACTTGTGATCAAGGT
AAAAGAGGTAGCAAAACCGTTCATACAAAGAAAATTGTCGATGAAGGTTT
AAATGGAATTTCTCAAGTTTCTCATATTCTTGTCTTCCAAAGGACAGGTG
CTGAAGGGATCCCAATGACACCTGGCAGAGATTACTGGTGGCACGAAGAA
GCTGCTAAGCAAAGAGGTTACATTCCACCAGTTCCTTGTAGTGCTGAAGA
TCCATTATTCCTCTTGTACACTTCAGGTTCCACCGGGTCACCAAAGGGTG
TGGTCCATTCAACCGGTGGTTATCTATTGGGTGCAGCCATGACCACTAGA
TATGTGTTTGACATCCATCCAGAAGATGTCTTATTCACAGCAGGTGATGT
TGGTTGGATTACAGGTCATACTTATGCTCTTTATGGTCCATTAGCGCTAG
GTACTGCATCAATCATCTTTGAATCAACACCAGCTTATCCTGATTACGGT
AGATATTGGAGAATCATTCAGCGTCATAAGGCAACTCATTTCTACGTGGC
TCCAACAGCCATGAGATTGATTAAGCGTGTAGGTGAGGCTGAAATTCCAA
AATACGATCTATCTTCACTAAGAGTTCTTGGATCAGTCGGTGAACCAATT
TCACCAGATCTTTGGGAATGGTACAACGAAAAAATTGGTCACAACAACTG
TGTCGTTTGTGATACCATGTGGCAAACCGAATCTGGTTCTCATTTAATTG
CTCCATTAGCAGGTGCAGTCCCAACAAAACCTGGTTCCGCTACAGTTCCA
TTCTTTGGTGTTGATGCTTGTATCATTGACCCAGTCACTGGTGTTGAATT
ACAAGGTAATGACGTGGAAGGTGTTTTGGCAGTAAAATCATCTTGGCCAT
CTATGGCAAGATCAGTTTGGCAAAATCACAACCGTTTCCAGGAGACTTAC
TTGCAACCATACCCTGGTTACTACTTTACAGGTGATGGTGCAGGTAGAGA
CCATGATGGTTACTACTGGATCAGAGGTAGAGTTGATGATGTGGTTAACG
TTTCTGGCCACAGATTGTCCACTGCTGAAATTGAAGCTTCATTGACCAAC
CATGATAATGTTTCTGAATCTGCTGTCGTAGGTATTCCTGACGAATTGAC
CGGTCAAACCGTCATTGCCTTCGTTGCATTGAAAGATGGTACTCCAAGTC
AAGGTGATGCGAGTGCTAACGTTCGTCGTGAATTGGTGCTCCAAGTTAGA
GGTGAAATTGGTCCATTTGCTGCTCCTAAATGTGTCATCTTGGTTAAAGA
TCTGCCAAAGACTAGATCCGGTAAAATCATGAGAAGAGTCCTAAGAAAAG
TTGCATCTAATGAAGCTGATCAACTGGGTGATCTATCTACTATGGCTAAC
GCTGAAGTCGTTCCAGGTATCATTGCAGCTGTTGATGAACAATATTTTGC
TGAGAAGAAGAAATAA

Amino acid sequence of the ACS2p from Z. rouxii strain CBS762 (SEQ ID NO: 6):

MTTKEHKTVHEAQNVVARHAPEHFYKSQPGLGYVKDMKQYQEMYKQSVED
PETFFGTKAQELLHWDKPFTKTKFGSLENGDTTWFLNGELNAAYNCVDRH
AFANPDKPALIYEADEEADNRVVTFGELLRQVSQVAGVLHSWGVRKGDTV
AVYLPMIPEAVVAMLAVARLGAIHSVVFAGFSAGSLKDRVVDAGCKVVIT
CDQGKRGSKTVHTKKIVDEGLNGISQVSHILVFQRTGAEGIPMTPGRDYW
WHEEAAKQRGYIPPVPCSAEDPLFLLYTSGSTGSPKGVVHSTGGYLLGAA
MTTRYVFDIHPEDVLFTAGDVGWITGHTYALYGPLALGTASIIFESTPAY
PDYGRYWRIIQRHKATHFYVAPTAMRLIKRVGEAEIPKYDLSSLRVLGSV
GEPISPDLWEWYNEKIGHNNCVVCDTMWQTESGSHLIAPLAGAVPTKPGS
ATVPFFGVDACIIDPVTGVELQGNDVEGVLAVKSSWPSMARSVWQNHNRF
QETYLQPYPGYYFTGDGAGRDHDGYYWIRGRVDDVVNVSGHRLSTAEIEA
SLTNHDNVSESAVVGIPDELTGQTVIAFVALKDGTPSQGDASANVRRELV
LQVRGEIGPFAAPKCVILVKDLPKTRSGKIMRRVLRKVASNEADQLGDLS
TMANAEVVPGIIAAVDEQYFAEKKK

Synthetic, yeast adopted DNA sequence encoding Salmonella typhimurium ACS L641P mutant (SEQ ID NO: 7):

ATGAACCAACAAGACATAGAACAAGTAGTAAAAGCCGTATTATTAAAGAT
GAAAGACTCCTCTCAACCAGCCTCAACCGTACACGAAATGGGTGTTTTTG
CCTCTTTGGATGACGCTGTCGCTGCAGCCAAAAGAGCCCAACAAGGTTTG
AAGTCAGTTGCTATGAGACAATTAGCAATCCATGCCATTAGAGAAGCAGG
TGAAAAACACGCCAGAGAATTGGCTGAATTAGCAGTATCCGAAACTGGTA
TGGGTAGAGTTGATGACAAATTCGCTAAGAATGTCGCTCAAGCAAGAGGT
ACACCAGGTGTCGAATGTTTGAGTCCTCAAGTATTAACAGGTGACAATGG
TTTGACCTTAATTGAAAACGCCCCATGGGGTGTTGTCGCTTCTGTTACAC
CATCAACCAATCCTGCTGCAACTGTTATAAATAACGCAATCTCTTTGATC
GCCGCTGGTAACTCAGTAGTTTTTGCTCCACATCCTGCAGCCAAAAAGGT
TTCCCAAAGAGCAATTACATTGTTAAATCAAGCCGTCGTAGCTGCAGGTG
GTCCAGAAAATTTGTTAGTAACCGTTGCTAACCCTGATATCGAAACTGCA
CAAAGATTATTCAAGTATCCAGGTATCGGTTTGTTAGTTGTCACAGGTGG
TGAAGCTGTAGTTGATGCCGCTAGAAAACACACCAATAAGAGATTGATTG
CAGCCGGTGCAGGTAACCCACCTGTCGTAGTTGATGAAACTGCTGACTTA
CCAAGAGCTGCACAATCCATCGTTAAGGGTGCAAGTTTCGATAACAACAT
CATCTGCGCTGACGAAAAGGTTTTATTGTCGTAGATTCTGTCGCTGACGA
ATTGATGAGATTAATGGAAGGTCAACATGCAGTTAAATTGACAGCCGCTC
AAGCCGAACAATTGCAACCAGTTTTGTTGAAAAATATAGATGAACGTGGT
AAAGGTACCGTATCAAGAGATTGGGTTGGTAGAGACGCAGGTAAAATTGC
AGCCGCTATAGGTTTGAACGTTCCTGATCAAACTAGATTGTTGTTCGTTG
AAACACCAGCTAACCATCCTTTCGCAGTAACAGAAATGATGATGCCAGTT
TTACCTGTTGTCAGAGTTGCTAATGTCGAAGAAGCCATAGCTTTGGCAGT
TCATTAGAAGGTGGTTGTCATCACACCGCAGCCATGCACTCCAGAAATAT
CGATAATATGAACCAAATGGCCAACGCTATCGACACTTCTATTTTCGTTA
AAAACGGTCCATGCATTGCTGGTTTGGGTTTAGGTGGTGAAGGTTGGACT
ACAATGACCATAACCACTCCTACTGGTGAAGGTGTCACTTCTGCAAGAAC
ATTTGTAAGATTGAGAAGATGTGTCTTAGTAGATGCTTTCAGAATTGTTT
AG

Amino acid sequence of the Salmonella typhimurium ACS L641P mutant (SEQ ID NO: 8):

MSQTHKHAIPANIADRCLINPEQYETKYKQSINDPDTFWGEQGKILDWIT
PYQKVKNTSFAPGNVSIKWYEDGTLNLAANCLDRHLQENGDRTAIIWEGD
DTSQSKHISYRELHRDVCRFANTLLDLGIKKGDVVAIYMPMVPEAAVAML
ACARIGAVHSVIFGGFSPEAVAGRIIDSSSRLVITADEGVRAGRSIPLKK
NVDDALKNPNVTSVEHVIVLKRTGSDIDWQEGRDLWWRDLIEKASPEHQP
EAMNAEDPLFILYTSGSTGKPKGVLHTTGGYLVYAATTFKYVFDYHPGDI
YWCTADVGWVTGHSYLLYGPLACGATTLMFEGVPNWPTPARMCQVVDKHQ
VNILYTAPTAIRALMAEGDKAIEGTDRSSLRILGSVGEPINPEAWEWYWK
KIGKEKCPVVDTWWQTETGGFMITPLPGAIELKAGSATRPFFGVQPALVD
NEGHPQEGATEGNLVITDSWPGQARTLFGDHERFEQTYFSTFKNMYFSGD
GARRDEDGYYWITGRVDDVLNVSGHRLGTAEIESALVAHPKIAEAAVVGI
PHAIKGQAIYAYVTLNHGEEPSPELYAEVRNWVRKEIGPLATPDVLHWTD
SLPKTRSGKIMRRILRKIAAGDTSNLGDTSTLADPGVVEKPLEEKQAIAM
PS

Synthetic, yeast adopted DNA sequence encoding Methanosaeta concilii acsAlp (SEQ ID NO: 9):

ATGTTGAAGTTGGCCGGTAAAGAAGATAAGAAGTTGAAAACCACTGTTTT
CCAAGACGAAACCAGAATTTTCAACCCACCAAAAGAATTGGTCGAAAAGT
CCATAGTTATGCAATGGATGAAGAAGAAGGGTTTCAAGACCGAAAAAGAA
ATGAGAGCTTGGTGTTCCTCTGATGAACACTATTTGGAATTTTGGGACGA
AATGGCTAAGACCTATGTTGATTGGCATAAGACTTACACCAAGGTTATGG
ATGATTCCGAAATGCCATACTTCCATTGGTTTACTGGTGGTGAAATCAAC
ATTACCTACAACGCTGTTGATAGACATGCTAAAGGTGCTAAGAAAGATAA
GGTTGCCTACATCTGGATTCCAGAACCTACTGATCAACCAGTTCAAAAGA
TTACTTACGGTGACTTGTACAAAGAAGTCAACAAGTTTGCTAACGGTTTG
AAGTCTTTGGGTTTGAAAAAGGGTGACAGAGTCTCTATCTACATGCCAAT
GATTCCACAATTGCCAATTGCTATGTTGGCTTGTGCTAAGTTGGGTGTTA
TTCACTCTGTTGTTTTCTCCGGTTTTTCCAGTAAAGGTTTGATGGATAGA
GCTGCTGATTGTGGTTCAAGAGCTATTATTACTGTTGACGGTTTCTACAG
AAGAGGTAAACCAGTTCCATTGAAGCCAAATGCTGATGAAGCTGCTGGTG
GTGCTCCATCTGTTGAAAAGATTATCGTTTACAAAAGAGCCGGTGTCGAT
GTCTCTATGAAGGAAGGTAGAGATGTTTGGTGGCATGATTTGGTTAAGGG
TCAATCTGAAGAATGTGAACCAGTTTGGGTTGATCCAGAACATAGATTGT
ACATCTTGTACACCTCTGGTACTACTGGTAAGCCAAAAGGTATTGAACAT
GCAACTGGTGGTAATGCTGTTGGTCCAGCTCAAACTTTACATTGGGTTTT
CGATTTGAAGGATGATGATGTATGGTGGTGTACTGCTGATATTGGTTGGG
TTACTGGTCATTCTTACATCGTTTATGCCCCATTGATTTTGGGTATGACC
TCTTTGATGTATGAAGGTGCTGCAGATTATCCAGATTTTGGTAGATGGTG
GAAGAACATCCAAGATCATAAGGTTACTGTCTTGTATACTGCTCCAACTG
CTGTTAGAATGTTCATGAAGCAAGGTGCTGAATGGCCAGATAAGTATGAT
TTGTCCTCCTTGAGATTATTGGGTTCTGTTGGTGAACCTATTAACCCTGA
AGCCTGGATGTGGTATAGAGAACATATTGGTAGAGGTGAATTGCAAATCA
TGGATACTTGGTGGCAAACTGAAACCGGTACTTTTTTGAACTCTCCATTG
CCTATTACCCCATTGAAACCAGGTTCTTGTACTTTTCCATTGCCAGGTTA
CGATATCTCCATTTTGGACGAAGAAGGTAACGAAGTTCCATTAGGTTCAG
GTGGTAATATCGTTGCTTTGAAACCATACCCATCTATGTTGAGAGCTTTT
TGGGGTGACAAAGAAAGATTCATGAAGGAATACTGGCAATTCTACTGGGA
TGTTCCAGGTAGAAGAGGTGTTTATTTGGCTGGTGATAAGGCTCAAAGAG
ATAAGGACGGTTACTTCTTCATTCAAGGTAGAATCGATGATGTTTTGTCC
GTTGCTGGTCATAGAATTGCTAATGCTGAAGTTGAATCTGCTTTGGTTGC
TCATCCAAAAATTGCTGAAGCTGCAGTTGTTGGTAAACCTGATGAAGTAA
AAGGTGAATCTATCGTTGCCTTCGTTATCTTGAGAGTTGGTAATGAACCA
TCTCCAGAATTGGCTAAAGATGCCATTGCTTTCGTTAGAAAAACTTTGGG
TCCAGTTGCTGCTCCTACTGAAGTTCATTTTGTTAACGATTTGCCAAAGA
CTAGATCCGGTAAGATCATGAGAAGAGTTGTTAAGGCTAGAGCTTTGGGT
AATCCAGTTGGTGATATTTCCACTTTGATGAATCCTGAAGCCGTTGATGG
TATTCCAAAGATCGTTTAA

Amino acid sequence of the Methanosaeta concilii acsAlp (SEQ ID NO: 10):

MLKLAGKEDKKLKTTVFQDETRIFNPPKELVEKSIVMQWMKKKGFKTEKE
MRAWCSSDEHYLEFWDEMAKTYVDWHKTYTKVMDDSEMPYFHWFTGGEIN
ITYNAVDRHAKGAKKDKVAYIWIPEPTDQPVQKITYGDLYKEVNKFANGL
KSLGLKKGDRVSIYMPMIPQLPIAMLACAKLGVIHSVVFSGFSSKGLMDR
AADCGSRAIITVDGFYRRGKPVPLKPNADEAAGGAPSVEKIIVYKRAGVD
VSMKEGRDVWWHDLVKGQSEECEPVWVDPEHRLYILYTSGTTGKPKGIEH
ATGGNAVGPAQTLHWVFDLKDDDVWWCTADIGWVTGHSYIVYAPLILGMT
SLMYEGAADYPDFGRWWKNIQDHKVTVLYTAPTAVRMFMKQGAEWPDKYD
LSSLRLLGSVGEPINPEAWMWYREHIGRGELQIMDTWWQTETGTFLNSPL
PITPLKPGSCTFPLPGYDISILDEEGNEVPLGSGGNIVALKPYPSMLRAF
WGDKERFMKEYWQFYWDVPGRRGVYLAGDKAQRDKDGYFFIQGRIDDVLS
VAGHRIANAEVESALVAHPKIAEAAVVGKPDEVKGESIVAFVILRVGNEP
SPELAKDAIAFVRKTLGPVAAPTEVHFVNDLPKTRSGKIMRRVVKARALG
NPVGDISTLMNPEAVDGIPKIV

What is claimed is:

1. Modified yeast cells, comprising:
(i) an exogenous gene encoding an acetyl-Co synthase enzyme derived from an archaeal microorganism belonging to the genus Methanosaeta,
(ii) an exogenous gene encoding a low-affinity acetyl-Co synthase enzyme derived from ascomycete yeast,
(iii) an overexpressed endogenous low-affinity acetyl-CoA synthase, and/or
(iv) an exogenous gene encoding a functionally similar protein having at least 80% amino acid sequence identity to a polypeptide of SEQ ID NO: 4, SEQ ID NO:6 or SEQ ID NO: 10, or an active fragment thereof,
wherein the modified cells produce a reduced amount of acetate and/or grow in the presence of an increased amount of acetate, compared to otherwise identical yeast cells not comprising the exogenous gene.

2. The modified yeast cells of claim 1, further comprising a genetic modification that, in the absence of the exogenous gene, causes the yeast cells to produce an increased amount of acetate.

3. The modified yeast cells of claim 2, wherein the genetic modification is the introduction of one or more genes of the phosphoketolase pathway or the deletion or disruption of genes of the glycerol synthesis pathway.

4. The modified yeast cells of any of the preceding claims, wherein the reduction in the amount of acetate produced is at least 20%, at least 30% or at least 40%.

5. The modified yeast cells of any of the preceding claims, wherein the functionally similar protein has at least 90%, at least 95% or at least 97% amino acid sequence identity to a polypeptide of SEQ ID NO: 3 or SEQ ID NO: 9, or an active fragment thereof.

6. The modified yeast cells of any of the preceding claims, further comprising a gene encoding a carbohydrate processing enzyme or other protein of interest.

7. A method for reducing the amount of acetate produced by yeast cells during a fermentation of a starch hydrolysate, comprising, introducing into the yeast:
(i) an exogenous gene encoding an acetyl-Co synthase enzyme derived from an archaeal microorganism belonging to the genus Methanosaeta,
(ii) an exogenous gene encoding a “low-affinity” acetyl-Co synthase enzyme derived from ascomycete yeast,
(iii) a genetic alteration that causes the overexpression of an endogenous low-affinity acetyl-CoA synthase, and/or
(iv) an exogenous gene encoding a functionally similar protein having at least 80% amino acid sequence identity to a polypeptide of SEQ ID NO: 3 or SEQ ID NO: 9, or an active fragment thereof.

8. The method of claim 7, wherein the yeast cells further comprise a genetic modification that, in the absence of the exogenous gene, would cause the yeast cells to produce an increased amount of acetate.

9. The method of claim 8, wherein the genetic modification is the introduction of genes of the phosphoketolase pathway or the deletion or disruption of genes of the glycerol synthesis pathway.

10. The method of any of claims 7-9, wherein the reduction in the amount of acetate produced is at least 20%, at least 30% or at least 40%.

11. The method of any of claims 7-10, wherein the functionally similar protein has at least 90%, at least 95% or at least 97% amino acid sequence identity to a polypeptide of SEQ ID NO: 3 or SEQ ID NO: 9, or an active fragment thereof.

12. Modified yeast cells produced by the method of any of claims 7-11.


SEQUENCE LISTING (10 sequences)

SEQ ID NO: 1 - DNA, 2142 bp, Saccharomyces cerevisiae
atgtcgccctctgccgtacaatcatcaaaactagaagaacagtcaagtgaaattgacaagttgaaagcaaaaatgtcccagtctgcctccactgcgcagcagaagaaggaacatgagtatgaacatttgacctcggtcaagatcgtgccacaacggcccatctcagatagactgcagcccgcaattgctacccactattctccacacttggacgggttgcaggactatcagcgcttgcacaaggagtctattgaagaccctgctaagttcttcggttctaaagctacccaatttttaaactggtctaagccattcgataaggtgttcatcccagaccctaaaacgggcaggccctccttccagaacaatgcatggttcctcaacggccaattaaacgcctgttacaactgtgttgacagacatgccttgaagacccctaacaagaaagccattattttcgaaggtgacgagcctggccaaggctattccattacctacaaggaactacttgaagaagtttgtcaagtggcacaagtgctgacttactctatgggcgttcgcaagggcgatactgttgccgtgtacatgcctatggtcccagaagcaatcataaccttgttggccatttcccgtatcggtgccattcactccgtagtctttgccgggttttcttccaactccttgagagatcgtatcaacgatggggactctaaagttgtcatcactacagatgaatccaacagaggtggtaaagtcattgagactaaaagaattgttgatgacgcgctaagagagaccccaggcgtgagacacgtcttggtttatagaaagaccaacaatccatctgttgctttccatgcccccagagatttggattgggcaacagaaaagaagaaatacaagacctactatccatgcacacccgttgattctgaggatccattattcttgttgtatacgtctggttctactggtgcccccaagggtgttcaacattctaccgcaggttacttgctgggagctttgttgaccatgcgctacacttttgacactcaccaagaagacgttttcttcacagctggagacattggctggattacaggccacacttatgtggtttatggtcccttactatatggttgtgccactttggtctttgaagggacgcctgcatacccaaattactcccgttattgggatattattgatgaacacaaagtcacccaattttatgttgcgccaactgctttgcgtttgttgaaaagagctggtgattcctacatcgaaaatcattccttaaaatctttgcgttgcttgggttcggtcggtgagccaattgctgctgaagtttgggagtggtactctgaaaaaataggtaaaaatgaaatccccattgtagacacctactggcaaacagaatctggttcgcatctggtcaccccgctggctggtggtgtcacaccaatgaaaccgggttctgcctcattccccttcttcggtattgatgcagttgttcttgaccctaacactggtgaagaacttaacaccagccacgcagagggtgtccttgccgtcaaagctgcatggccatcatttgcaagaactatttggaaaaatcatgataggtatctagacacttatttgaacccttaccctggctactatttcactggtgatggtgctgcaaaggataaggatggttatatctggattttgggtcgtgtagacgatgtggtgaacgtctctggtcaccgtctgtctaccgctgaaattgaggctgctattatcgaagatccaattgtggccgagtgtgctgttgtcggattcaacgatgacttgactggtcaagcagttgctgcatttgtggtgttgaaaaacaaatctagttggtccaccgcaacagatgatgaattacaagatatcaagaagcatttggtctttactgttagaaaagacatcgggccatttgccgcaccaaaattgatcattttagtggatgacttgcccaagacaagatctggcaaaattatgagacgtattttaagaaaaatcctagcaggagaaagtgaccaactaggcgacgtttctacattgtcaaaccctggcattgttagacatctaattgattcggtcaagttgtaa

SEQ ID NO: 2 - protein, 713 aa, Saccharomyces cerevisiae
MSPSAVQSSKLEEQSSEIDKLKAKMSQSASTAQQKKEHEYEHLTSVKIVPQRPISDRLQPAIATHYSPHLDGLQDYQRLHKESIEDPAKFFGSKATQFLNWSKPFDKVFIPDPKTGRPSFQNNAWFLNGQLNACYNCVDRHALKTPNKKAIIFEGDEPGQGYSITYKELLEEVCQVAQVLTYSMGVRKGDTVAVYMPMVPEAIITLLAISRIGAIHSVVFAGFSSNSLRDRINDGDSKVVITTDESNRGGKVIETKRIVDDALRETPGVRHVLVYRKTNNPSVAFHAPRDLDWATEKKKYKTYYPCTPVDSEDPLFLLYTSGSTGAPKGVQHSTAGYLLGALLTMRYTFDTHQEDVFFTAGDIGWITGHTYVVYGPLLYGCATLVFEGTPAYPNYSRYWDIIDEHKVTQFYVAPTALRLLKRAGDSYIENHSLKSLRCLGSVGEPIAAEVWEWYSEKIGKNEIPIVDTYWQTESGSHLVTPLAGGVTPMKPGSASFPFFGIDAVVLDPNTGEELNTSHAEGVLAVKAAWPSFARTIWKNHDRYLDTYLNPYPGYYFTGDGAAKDKDGYIWILGRVDDVVNVSGHRLSTAEIEAAIIEDPIVAECAVVGFNDDLTGQAVAAFVVLKNKSSWSTATDDELQDIKKHLVFTVRKDIGPFAAPKLIILVDDLPKTRSGKIMRRILRKILAGESDQLGDVSTLSNPGIVRHLIDSVKL

SEQ ID NO: 3 - DNA, 2052 bp, Saccharomyces cerevisiae
atgacaatcaaggaacataaagtagttcatgaagctcacaacgtaaaggctcttaaggctcctcaacatttttacaacagccaacccggcaagggttacgttactgatatgcaacattatcaagaaatgtatcaacaatctatcaatgagccagaaaaattctttgataagatggctaaggaatacttgcattgggatgctccatacaccaaagttcaatctggttcattgaacaatggtgatgttgcatggtttttgaacggtaaattgaatgcatcatacaattgtgttgacagacatgcctttgctaatcccgacaagccagctttgatctatgaagctgatgacgaatccgacaacaaaatcatcacatttggtgaattactcagaaaagtttcccaaatcgctggtgtcttaaaaagctggggcgttaagaaaggtgacacagtggctatctatttgccaatgattccagaagcggtcattgctatgttggctgtggctcgtattggtgctattcactctgttgtctttgctgggttctccgctggttcgttgaaagatcgtgtcgttgacgctaattctaaagtggtcatcacttgtgatgaaggtaaaagaggtggtaagaccatcaacactaaaaaaattgttgacgaaggtttgaacggagtcgatttggtttcccgtatcttggttttccaaagaactggtactgaaggtattccaatgaaggccggtagagattactggtggcatgaggaggccgctaagcagagaacttacctacctcctgtttcatgtgacgctgaagatcctctatttttgttatacacttccggttccactggttctccaaagggtgtcgttcacactacaggtggttatttattaggtgccgctttaacaactagatacgtttttgatattcacccagaagatgttctcttcactgccggtgacgtcggctggatcacgggtcacacctatgctctatatggtccattaaccttgggtaccgcctcaataattttcgaatccactcctgcctacccagattatggtagatattggagaattatccaacgtcacaaggctacccatttctatgtggctccaactgctttaagattaatcaaacgtgtaggtgaagccgaaattgccaaatatgacacttcctcattacgtgtcttgggttccgtcggtgaaccaatctctccagacttatgggaatggtatcatgaaaaagtgggtaacaaaaactgtgtcatttgtgacactatgtggcaaacagagtctggttctcatttaattgctcctttggcaggtgctgtcccaacaaaacctggttctgctaccgtgccattctttggtattaacgcttgtatcattgaccctgttacaggtgtggaattagaaggtaatgatgtcgaaggtgtccttgccgttaaatcaccatggccatcaatggctagatctgtttggaaccaccacgaccgttacatggatacttacttgaaaccttatcctggtcactatttcacaggtgatggtgctggtagagatcatgatggttactactggatcaggggtagagttgacgacgttgtaaatgtttccggtcatagattatccacatcagaaattgaagcatccatctcaaatcacgaaaacgtctcggaagctgctgttgtcggtattccagatgaattgaccggtcaaaccgtcgttgcatatgtttccctaaaagatggttatctacaaaacaacgctactgaaggtgatgcagaacacatcacaccagataatttacgtagagaattgatcttacaagttaggggtgagattggtcctttcgcctcaccaaaaaccattattctagttagagatctaccaagaacaaggtcaggaaagattatgagaagagttctaagaaaggttgcttctaacgaagccgaacagctaggtgacctaactactttggccaacccagaagttgtacctgccatcatttctgctgtagagaaccaatttttctctcaaaaaaagaaataa

SEQ ID NO: 4 - protein, 683 aa, Saccharomyces cerevisiae
MTIKEHKVVHEAHNVKALKAPQHFYNSQPGKGYVTDMQHYQEMYQQSINEPEKFFDKMAKEYLHWDAPYTKVQSGSLNNGDVAWFLNGKLNASYNCVDRHAFANPDKPALIYEADDESDNKIITFGELLRKVSQIAGVLKSWGVKKGDTVAIYLPMIPEAVIAMLAVARIGAIHSVVFAGFSAGSLKDRVVDANSKVVITCDEGKRGGKTINTKKIVDEGLNGVDLVSRILVFQRTGTEGIPMKAGRDYWWHEEAAKQRTYLPPVSCDAEDPLFLLYTSGSTGSPKGVVHTTGGYLLGAALTTRYVFDIHPEDVLFTAGDVGWITGHTYALYGPLTLGTASIIFESTPAYPDYGRYWRIIQRHKATHFYVAPTALRLIKRVGEAEIAKYDTSSLRVLGSVGEPISPDLWEWYHEKVGNKNCVICDTMWQTESGSHLIAPLAGAVPTKPGSATVPFFGINACIIDPVTGVELEGNDVEGVLAVKSPWPSMARSVWNHHDRYMDTYLKPYPGHYFTGDGAGRDHDGYYWIRGRVDDVVNVSGHRLSTSEIEASISNHENVSEAAVVGIPDELTGQTVVAYVSLKDGYLQNNATEGDAEHITPDNLRRELILQVRGEIGPFASPKTIILVRDLPRTRSGKIMRRVLRKVASNEAEQLGDLTTLANPEVVPAIISAVENQFFSQKKK

SEQ ID NO: 5 - DNA, 1866 bp, Zymomonis rouxii
atgacaactaaggaacataagaccgttcatgaggataaaccatttacaaagacgaagtttggttcattggaaaatggtgatactacttggtttttaaacggtgagttgaatgctgcttacaactgtgttgatagacatgcttttgccaatcctgataaaccagcattgatctacgaagcggatgaagaagcagacaacagggttgttacattcggtgagcttttgagacaagtttctcaagttgctggtgttcttcacagctggggggttagaaaaggtgataccgtcgcagtttatctaccaatgattcctgaagctgttgttgcaatgttggccgttgccagattaggtgcaattcattctgttgtttttgcaggtttttcagcaggttccttgaaggatcgtgttgtagacgcaggttgtaaagttgttattacttgtgatcaaggtaaaagaggtagcaaaaccgttcatacaaagaaaattgtcgatgaaggtttaaatggaatttctcaagtttctcatattcttgtcttccaaaggacaggtgctgaagggatcccaatgacacctggcagagattactggtggcacgaagaagctgctaagcaaagaggttacattccaccagttccttgtagtgctgaagatccattattcctcttgtacacttcaggttccaccgggtcaccaaagggtgtggtccattcaaccggtggttatctattgggtgcagccatgaccactagatatgtgtttgacatccatccagaagatgtcttattcacagcaggtgatgttggttggattacaggtcatacttatgctctttatggtccattagcgctaggtactgcatcaatcatctttgaatcaacaccagcttatcctgattacggtagatattggagaatcattcagcgtcataaggcaactcatttctacgtggctccaacagccatgagattgattaagcgtgtaggtgaggctgaaattccaaaatacgatctatcttcactaagagttcttggatcagtcggtgaaccaatttcaccagatctttgggaatggtacaacgaaaaaattggtcacaacaactgtgtcgtttgtgataccatgtggcaaaccgaatctggttctcatttaattgctccattagcaggtgcagtcccaacaaaacctggttccgctacagttccattctttggtgttgatgcttgtatcattgacccagtcactggtgttgaattacaaggtaatgacgtggaaggtgttttggcagtaaaatcatcttggccatctatggcaagatcagtttggcaaaatcacaaccgtttccaggagacttacttgcaaccataccctggttactactttacaggtgatggtgcaggtagagaccatgatggttactactggatcagaggtagagttgatgatgtggttaacgtttctggccacagattgtccactgctgaaattgaagcttcattgaccaaccatgataatgtttctgaatctgctgtcgtaggtattcctgacgaattgaccggtcaaaccgtcattgccttcgttgcattgaaagatggtactccaagtcaaggtgatgcgagtgctaacgttcgtcgtgaattggtgctccaagttagaggtgaaattggtccatttgctgctcctaaatgtgtcatcttggttaaagatctgccaaagactagatccggtaaaatcatgagaagagtcctaagaaaagttgcatctaatgaagctgatcaactgggtgatctatctactatggctaacgctgaagtcgttccaggtatcattgcagctgttgatgaacaatattttgctgagaagaagaaataa

SEQ ID NO: 6 - protein, 675 aa, Zymomonis rouxii
MTTKEHKTVHEAQNVVARHAPEHFYKSQPGLGYVKDMKQYQEMYKQSVEDPETFFGTKAQELLHWDKPFTKTKFGSLENGDTTWFLNGELNAAYNCVDRHAFANPDKPALIYEADEEADNRVVTFGELLRQVSQVAGVLHSWGVRKGDTVAVYLPMIPEAVVAMLAVARLGAIHSVVFAGFSAGSLKDRVVDAGCKVVITCDQGKRGSKTVHTKKIVDEGLNGISQVSHILVFQRTGAEGIPMTPGRDYWWHEEAAKQRGYIPPVPCSAEDPLFLLYTSGSTGSPKGVVHSTGGYLLGAAMTTRYVFDIHPEDVLFTAGDVGWITGHTYALYGPLALGTASIIFESTPAYPDYGRYWRIIQRHKATHFYVAPTAMRLIKRVGEAEIPKYDLSSLRVLGSVGEPISPDLWEWYNEKIGHNNCVVCDTMWQTESGSHLIAPLAGAVPTKPGSATVPFFGVDACIIDPVTGVELQGNDVEGVLAVKSSWPSMARSVWQNHNRFQETYLQPYPGYYFTGDGAGRDHDGYYWIRGRVDDVVNVSGHRLSTAEIEASLTNHDNVSESAVVGIPDELTGQTVIAFVALKDGTPSQGDASANVRRELVLQVRGEIGPFAAPKCVILVKDLPKTRSGKIMRRVLRKVASNEADQLGDLSTMANAEVVPGIIAAVDEQYFAEKKK

SEQ ID NO: 7 - DNA, 1404 bp, Salmonella typhimurium
atgaaccaacaagacatagaacaagtagtaaaagccgtattattaaagatgaaagactcctctcaaccagcctcaaccgtacacgaaatgggtgtttttgcctctttggatgacgctgtcgctgcagccaaaagagcccaacaaggtttgaagtcagttgctatgagacaattagcaatccatgccattagagaagcaggtgaaaaacacgccagagaattggctgaattagcagtatccgaaactggtatgggtagagttgatgacaaattcgctaagaatgtcgctcaagcaagaggtacaccaggtgtcgaatgtttgagtcctcaagtattaacaggtgacaatggtttgaccttaattgaaaacgccccatggggtgttgtcgcttctgttacaccatcaaccaatcctgctgcaactgttataaataacgcaatctctttgatcgccgctggtaactcagtagtttttgctccacatcctgcagccaaaaaggtttcccaaagagcaattacattgttaaatcaagccgtcgtagctgcaggtggtccagaaaatttgttagtaaccgttgctaaccctgatatcgaaactgcacaaagattattcaagtatccaggtatcggtttgttagttgtcacaggtggtgaagctgtagttgatgccgctagaaaacacaccaataagagattgattgcagccggtgcaggtaacccacctgtcgtagttgatgaaactgctgacttaccaagagctgcacaatccatcgttaagggtgcaagtttcgataacaacatcatctgcgctgacgaaaaggttttaattgtcgtagattctgtcgctgacgaattgatgagattaatggaaggtcaacatgcagttaaattgacagccgctcaagccgaacaattgcaaccagttttgttgaaaaatatagatgaacgtggtaaaggtaccgtatcaagagattgggttggtagagacgcaggtaaaattgcagccgctataggtttgaacgttcctgatcaaactagattgttgttcgttgaaacaccagctaaccatcctttcgcagtaacagaaatgatgatgccagttttacctgttgtcagagttgctaatgtcgaagaagccatagctttggcagttcaattagaaggtggttgtcatcacaccgcagccatgcactccagaaatatcgataatatgaaccaaatggccaacgctatcgacacttctattttcgttaaaaacggtccatgcattgctggtttgggtttaggtggtgaaggttggactacaatgaccataaccactcctactggtgaaggtgtcacttctgcaagaacatttgtaagattgagaagatgtgtcttagtagatgctttcagaattgtttag

SEQ ID NO: 8 - protein, 652 aa, Salmonella typhimurium
MSQTHKHAIPANIADRCLINPEQYETKYKQSINDPDTFWGEQGKILDWITPYQKVKNTSFAPGNVSIKWYEDGTLNLAANCLDRHLQENGDRTAIIWEGDDTSQSKHISYRELHRDVCRFANTLLDLGIKKGDVVAIYMPMVPEAAVAMLACARIGAVHSVIFGGFSPEAVAGRIIDSSSRLVITADEGVRAGRSIPLKKNVDDALKNPNVTSVEHVIVLKRTGSDIDWQEGRDLWWRDLIEKASPEHQPEAMNAEDPLFILYTSGSTGKPKGVLHTTGGYLVYAATTFKYVFDYHPGDIYWCTADVGWVTGHSYLLYGPLACGATTLMFEGVPNWPTPARMCQVVDKHQVNILYTAPTAIRALMAEGDKAIEGTDRSSLRILGSVGEPINPEAWEWYWKKIGKEKCPVVDTWWQTETGGFMITPLPGAIELKAGSATRPFFGVQPALVDNEGHPQEGATEGNLVITDSWPGQARTLFGDHERFEQTYFSTFKNMYFSGDGARRDEDGYYWITGRVDDVLNVSGHRLGTAEIESALVAHPKIAEAAVVGIPHAIKGQAIYAYVTLNHGEEPSPELYAEVRNWVRKEIGPLATPDVLHWTDSLPKTRSGKIMRRILRKIAAGDTSNLGDTSTLADPGVVEKPLEEKQAIAMPS

SEQ ID NO: 9 - DNA, 2019 bp, Methanosaeta concilii
atgttgaagttggccggtaaagaagataagaagttgaaaaccactgttttccaagacgaaaccagaattttcaacccaccaaaagaattggtcgaaaagtccatagttatgcaatggatgaagaagaagggtttcaagaccgaaaaagaaatgagagcttggtgttcctctgatgaacactatttggaattttgggacgaaatggctaagacctatgttgattggcataagacttacaccaaggttatggatgattccgaaatgccatacttccattggtttactggtggtgaaatcaacattacctacaacgctgttgatagacatgctaaaggtgctaagaaagataaggttgcctacatctggattccagaacctactgatcaaccagttcaaaagattacttacggtgacttgtacaaagaagtcaacaagtttgctaacggtttgaagtctttgggtttgaaaaagggtgacagagtctctatctacatgccaatgattccacaattgccaattgctatgttggcttgtgctaagttgggtgttattcactctgttgttttctccggtttttccagtaaaggtttgatggatagagctgctgattgtggttcaagagctattattactgttgacggtttctacagaagaggtaaaccagttccattgaagccaaatgctgatgaagctgctggtggtgctccatctgttgaaaagattatcgtttacaaaagagccggtgtcgatgtctctatgaaggaaggtagagatgtttggtggcatgatttggttaagggtcaatctgaagaatgtgaaccagtttgggttgatccagaacatagattgtacatcttgtacacctctggtactactggtaagccaaaaggtattgaacatgcaactggtggtaatgctgttggtccagctcaaactttacattgggttttcgatttgaaggatgatgatgtatggtggtgtactgctgatattggttgggttactggtcattcttacatcgtttatgccccattgattttgggtatgacctctttgatgtatgaaggtgctgcagattatccagattttggtagatggtggaagaacatccaagatcataaggttactgtcttgtatactgctccaactgctgttagaatgttcatgaagcaaggtgctgaatggccagataagtatgatttgtcctccttgagattattgggttctgttggtgaacctattaaccctgaagcctggatgtggtatagagaacatattggtagaggtgaattgcaaatcatggatacttggtggcaaactgaaaccggtacttttttgaactctccattgcctattaccccattgaaaccaggttcttgtacttttccattgccaggttacgatatctccattttggacgaagaaggtaacgaagttccattaggttcaggtggtaatatcgttgctttgaaaccatacccatctatgttgagagctttttggggtgacaaagaaagattcatgaaggaatactggcaattctactgggatgttccaggtagaagaggtgtttatttggctggtgataaggctcaaagagataaggacggttacttcttcattcaaggtagaatcgatgatgttttgtccgttgctggtcatagaattgctaatgctgaagttgaatctgctttggttgctcatccaaaaattgctgaagctgcagttgttggtaaacctgatgaagtaaaaggtgaatctatcgttgccttcgttatcttgagagttggtaatgaaccatctccagaattggctaaagatgccattgctttcgttagaaaaactttgggtccagttgctgctcctactgaagttcattttgttaacgatttgccaaagactagatccggtaagatcatgagaagagttgttaaggctagagctttgggtaatccagttggtgatatttccactttgatgaatcctgaagccgttgatggtattccaaagatcgtttaa

SEQ ID NO: 10 - protein, 672 aa, Methanosaeta concilii
MLKLAGKEDKKLKTTVFQDETRIFNPPKELVEKSIVMQWMKKKGFKTEKEMRAWCSSDEHYLEFWDEMAKTYVDWHKTYTKVMDDSEMPYFHWFTGGEINITYNAVDRHAKGAKKDKVAYIWIPEPTDQPVQKITYGDLYKEVNKFANGLKSLGLKKGDRVSIYMPMIPQLPIAMLACAKLGVIHSVVFSGFSSKGLMDRAADCGSRAIITVDGFYRRGKPVPLKPNADEAAGGAPSVEKIIVYKRAGVDVSMKEGRDVWWHDLVKGQSEECEPVWVDPEHRLYILYTSGTTGKPKGIEHATGGNAVGPAQTLHWVFDLKDDDVWWCTADIGWVTGHSYIVYAPLILGMTSLMYEGAADYPDFGRWWKNIQDHKVTVLYTAPTAVRMFMKQGAEWPDKYDLSSLRLLGSVGEPINPEAWMWYREHIGRGELQIMDTWWQTETGTFLNSPLPITPLKPGSCTFPLPGYDISILDEEGNEVPLGSGGNIVALKPYPSMLRAFWGDKERFMKEYWQFYWDVPGRRGVYLAGDKAQRDKDGYFFIQGRIDDVLSVAGHRIANAEVESALVAHPKIAEAAVVGKPDEVKGESIVAFVILRVGNEPSPELAKDAIAFVRKTLGPVAAPTEVHFVNDLPKTRSGKIMRRVVKARALGNPVGDISTLMNPEAVDGIPKIV